(19) United States
(12) Patent Application Publication
(10) Pub. No.: US 2009/0093808 A1
(43) Pub. Date: Apr. 9, 2009

(54) PARTIAL (NON-APICAL) PROSTATE ABLATION PROCEDURE AND DEVICE

(75) Inventor: Gregg A. VanDusseldorp, SR., Valparaiso, IN (US)

Correspondence Address: HARTMAN & HARTMAN, P.C., 552 EAST 700 NORTH, VALPARAISO, IN, 46383, US

(73) Assignee: Gregg A. VanDusseldorp & Associates, Inc., Valparaiso, IN (US)

(21) Appl. No.: 12/245,107

(22) Filed: Oct. 3, 2008

Related U.S. Application Data
(60) Provisional application No. 60/977,201, filed on Oct. 3, 2007.
(60) Provisional application No. 60/988,458, filed on Nov. 16, 2007.

(51) Int. Cl. A61B 18/14

(52) U.S. Cl. 606/41

(57) ABSTRACT

A device and procedure for performing partial non-apical transurethral ablation of the prostate. The device includes a first conductor member reciprocably extending from a distal end of a sheath, a cap disposed at the distal end of the first conductor member, at least two nonconducting members interconnecting the cap and the distal end of the sheath, and multiple flexible conductor members distally extending from the distal end of the sheath, along the first conductor member, and interconnected with the distal end of the first conductor member. The device is operable to expand the nonconducting members and the flexible conductor members to perform ablation of a lower region of the prostatic urethra while an upper (apex) region of the prostatic urethra is held apart from the lower region by the nonconducting members so that the upper (apex) region does not undergo ablation.

### CROSS REFERENCE TO RELATED APPLICATIONS

This application claims the benefit of U.S. Provisional Application Nos. 60/977,201 filed Oct. 3, 2007, and 60/988,458 filed Nov. 16, 2007, whose contents are incorporated herein by reference. In addition, this application is related to U.S. Pat. No. 6,673,071 to VanDusseldorp et al., the contents of which are incorporated herein by reference.

### BACKGROUND OF THE INVENTION

The present invention generally relates to surgical procedures, and more particularly to an ablation procedure and an electrosurgical probe for treating damaged, diseased or enlarged tissue of the prostate.

Ablation and resection are electrosurgical effects accomplished by applying a highly damped radio frequency (RF) current to tissue through an electrode in the form of an active (+) tip of an electrosurgical (electrocautery) probe, from which the RF current flows to a second ground (−) electrode. As it passes through tissue from the active tip to the ground electrode, the RF current resects (cuts), coagulates and/or ablates (desiccates) the tissue, depending on the type of probe and the RF power and wave length combinations used. RF electrosurgical probes are typically placed through a resectoscope, hysteroscope or other device, which is often equipped with a telescope so that the active tip of the probe is in direct view of the surgeon at all times. Irrigating solutions are commonly used as a distention medium and a coolant for the active tips of RF probes during electrosurgical procedures. Nonconductive irrigation solutions such as sorbitol (C6H14O6) are commonly used as they promote the flow of RF current through the tissue being cut, instead of dissipating the current as would a conductive solution such as saline.

Electrosurgical resection refers to procedures by which damaged, diseased or enlarged tissue is removed with an electrosurgical probe. An example is transurethral resection of the prostate (TURP), in which prostate tissue is removed by means of an RF probe (for example, a cutting loop) passed through the urethra by means of a resectoscope. This procedure has served as the historical treatment of benign prostate hypertrophy (BPH)), commonly known as “enlarged prostate,” and prostatitus. In contrast, electrosurgical ablation is a procedure by which an RF probe (for example, a roller) is used to ablate (dessicate) tissue, which eventually sloughs off instead of being immediately removed on contact with the probe. An example of an electrosurgical ablation procedure is endometrial ablation, which is an electrosurgical alternative treatment to hysterectomy in women with menorrhagia (abnormal uterine bleeding). Another example is transurethral ablation of the prostate (TUAP), in which prostate tissue is ablated by means of an electrocautery probe passed over a stylet/obturator or guide wire, through the prostatic urethra. As such, ablation probes and procedures differ from resection probes and procedures that remove tissue on contact, such as TURP, as well as other electrosurgical probes and procedures, for example, that perform vaporization of the prostate (TUVP).

Considerable surgical skills are necessary to perform TUAP, typically necessitating that the procedure be performed in a hospital or other surgical setting. Complications can arise due to the risk of damage to the apex of the prostrate, and particularly nerves (e.g., the obturator nerve) at the apex. The device has the further advantage of being compatible with the use of conductive and nonconductive irrigating solutions, allowing for the use of normal saline solutions and eliminating concerns for TURP syndrome, which encompasses various symptoms caused by the absorption of large volumes of irrigation fluid during TURP.

### BRIEF DESCRIPTION OF THE INVENTION

The present invention provides an ablation device, electrosurgical probe and procedure for performing transurethral ablation of the prostate (TUAP), and more particularly, a probe and procedure for performing what is termed herein “partial” (non-apical) transurethral prostate ablation that can be used to treat BPH in an office setting.

According to a first aspect of the invention, the ablation device generally includes a first conductor member reciprocably extending from a distal end of a sheath and having a distal end that extends distally from the distal end of the sheath, a nonconducting cap disposed at the distal end of the first conductor member and having a distal opening therein, at least two nonconducting members interconnecting the nonconducting cap and the distal end of the sheath, and multiple flexible conductor members distally extending from the distal end of the sheath, along the first conductor member, and interconnected with the distal end of the first conductor member. The first conductor member and the nonconducting cap define a central channel configured to enable an introducing device to be passed therethrough and out through the distal opening of the nonconducting cap. The device has a stowed position in which the first conductor member extends from the sheath and the nonconducting members and the flexible conductor members are parallel to the first conductor member. In the stowed position, the sheath is sized to be inserted into the urethra and the first conductor member, the nonconducting cap, the nonconducting members, and the flexible conductor members are sized to be inserted into the prostatic urethra. The sheath, the first conductor member, the nonconducting members, and the flexible conductor members are interconnected so that retraction of the first conductor member relative to the sheath causes the distal end of the first conductor member to move proximally toward the sheath and causes the nonconducting members and the flexible conductor member to expand radially outward and away from the first conductor member and away from each other. When the first conductor member is retracted to expand the nonconducting members and the flexible conductor member, the flexible conductor member is operative to perform ablation of a lower region of the prostatic urethra between 2:30 and 9:30 (on the clock face) while an upper (apex) region of the prostatic urethra between 10:00 and 2:00 (on the clock face) is held apart from the lower region by the nonconducting members so that the upper (apex) region does not undergo ablation.

According to a second aspect of the invention, the procedure includes inserting an ablation device within the prostatic urethra of the human body with the assistance of an introducing device, and then causing a current to flow through multiple conductor members of the ablation device to perform a controlled and selective electrosurgical ablation of a lower region of the prostatic urethra between 2:30 and 9:30 (on the clock face) and not an upper region of the prostatic urethra between 10:00 and 2:00 (on the clock face) by holding the upper region apart from the lower region with at least two nonconducting members through which current does not flow.

In view of the above, it can be seen that a significant advantage of this invention is that the ability to perform partial ablation of the prostate reduces the risk of damage to the apex of the prostrate, and particularly nerves (e.g., the obturator nerve) at the apex. The device of this invention is able to perform a partial (non-apical) ablation in a single procedure, such as by providing controlled electrosurgical ablation covering a limited radial area of the prostatic urethra inferior of the apex, instead of the generally non-selective ablation/removal of prostatic tissue preformed with prior art devices and procedures.

Other objects and advantages of this invention will be better appreciated from the following detailed description.

### BRIEF DESCRIPTION OF THE DRAWINGS

FIG. 1 is a side view of a partial ablation device with a probe end shown expanded in accordance with a preferred embodiment of the invention.

FIGS. 2 and 3 are detailed side and end views, respectively, of the expanded probe end of the device shown in FIG. 1.

FIG. 4 is a side view of the partial ablation device of FIG. 1 shown with the probe end collapsed.

FIGS. 5 and 6 are detailed side and end views, respectively, of the collapsed probe end of the device shown in FIG. 4.

FIG. 7 represents an end view of the expanded probe end of FIG. 3 within a prostatic urethra for performing a partial (non-apical) prostate ablation procedure in accordance with the present invention.

### DETAILED DESCRIPTION OF THE INVENTION

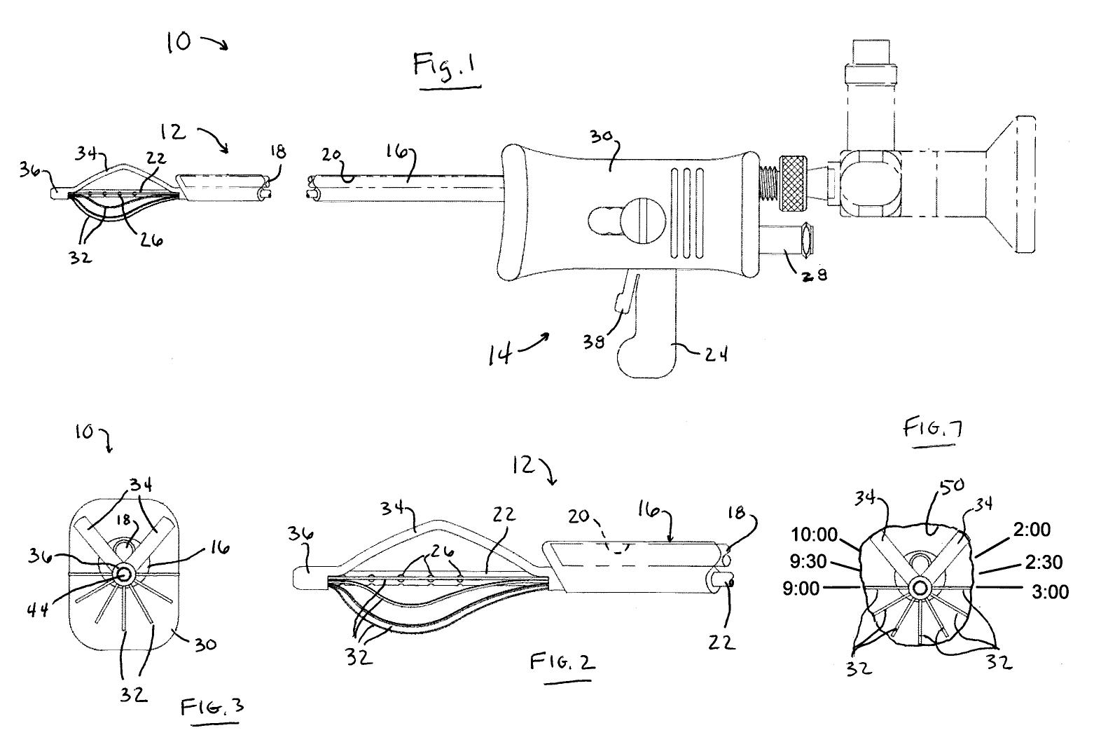

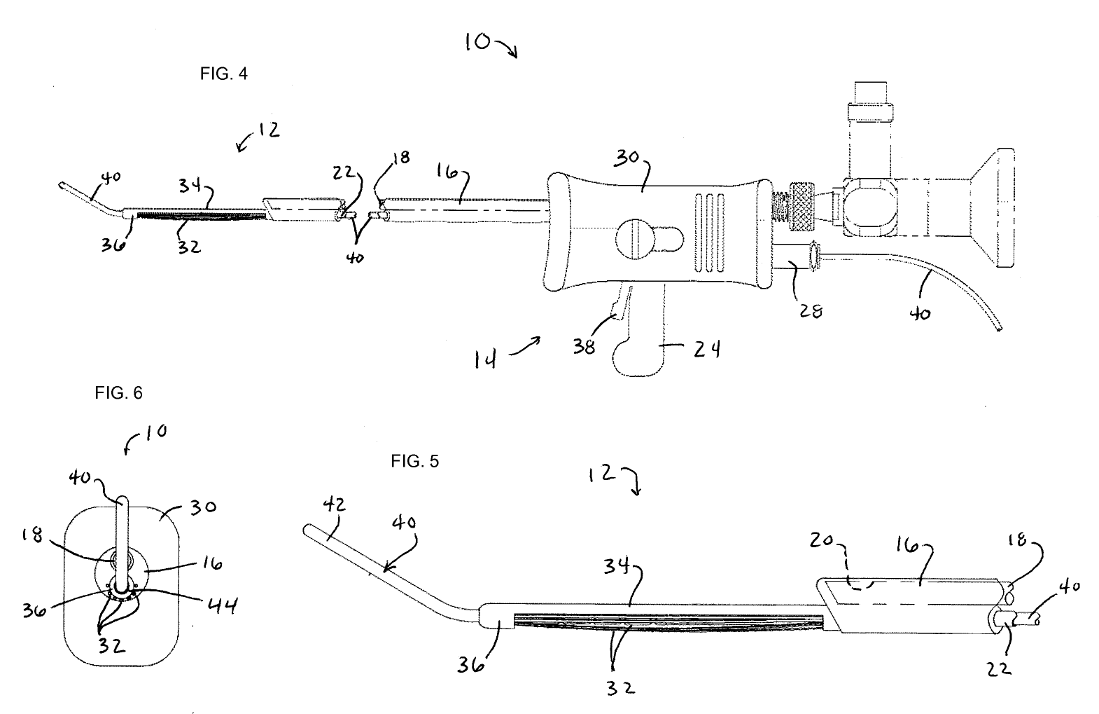

FIGS. 1 and 4 represent an electrosurgical ablation device 10 in accordance with a preferred embodiment of the present invention. In FIGS. 1 and 4, the device 10 is represented as having an RF probe 12 mounted to what will be termed a resectoscope 14, though it will be understood that the resectoscope 14 of FIGS. 1 and 4 differ from conventional resectoscopes, and particularly those used to perform resection. The device 10 and probe 12 will be discussed in particular reference to performing TUAP, and more particularly a partial non-apical prostate ablation procedure that can be used to treat BPH in an office setting. However, those skilled in the art will recognize that the device 10 and probe 12 may have other potential surgical uses.

The ablation device 10 is represented as including a sheath 16 mounted to a handle 30 of the resectoscope 14. The interior of the sheath 16 defines a channel 20 that receives a telescope 18 so that the probe 12 can be placed and the procedure performed under direct vision. The telescope 18 can be a conventional pediatric telescope or any other suitable design. Materials known and used for prior art ablation devices can be used to fabricate the sheath 12 and telescope 18.

A central conductor 22 is disposed within the sheath 16 so that the distal end of the conductor 22 extends outside the sheath 16. The conductor 22 is capable of reciprocal movement within the sheath 16 through the operation of an actuation lever 24. The conductor 22 preferably has an internal flow channel with a row of outlet ports 26 along its length to enable an irrigation fluid to be used. The handle 30 is represented as having a port 28 through which an irrigation fluid can be introduced into the flow channel of the central conductor 22. Various conductive materials can be used to form the central conductor 22, including AISI type 304 stainless steel.

The probe 12 is represented as comprising flexible electrically-conductive wires 32 and a pair of flexible nonconducting members 34. The proximal ends of the conductive wires 32 are anchored to the distal end of the sheath 16 so as to be radially spaced from the central conductor 22, as can be seen in FIG. 6. The wires 32 then extend from the distal end of the sheath 16 toward the distal end of the central conductor 22, to which the distal ends of the wires 32 are attached to form an electrical connection therewith. While shown as being formed of round wire, other cross-sections are foreseeable. Suitable materials for the wires 32 include tungsten and stainless steels, though other materials could also be used. The nonconducting members 34 interconnect the distal end of the central conductor 22 with the sheath 16. For this purpose, the central conductor 22 is shown as having a cap 36 on its distal end, with the nonconducting members 34 shown as being formed integral with the cap 36 and sheath 16. Accordingly, suitable nonconducting materials for the nonconducting members 34 and cap 36 are those suitable for the sheath 16. In comparing FIGS. 1 through 3 and 4 through 6, one can see that the probe 12 has a stowed position (FIGS. 4 through 6) in which the wires 32 and nonconducting members 34 are substantially parallel to the central conductor 22, and that retracting the central conductor 22 into the sheath 16 causes the wires 32 and nonconducting members 34 to be elastically displaced (expanded) radially outward away from the central conductor 22 in substantially opposite directions.

FIG. 3 shows seven conductive wires 32 equi-angularly spaced within an angular range of about 180 degrees, and two nonconducting members 34 spaced about 80 degrees apart in the remaining 180 range of the expanded probe 12. The number and spacing of the wires 32 and nonconducting members 34 is believed to be preferred for performing the partial prostate ablation of this invention, though it is foreseeable that other numbers and spacings of the wires and members 34 could be used. However, a minimum of seven wires 32 and a minimum of two nonconducting members 34 are believed necessary to properly distend the prostatic urethra 50 (FIG. 7) during the partial non-apical ablation procedure preferred by this invention. For use in treating the prostrate, the wires 32 and nonconducting members 34 are preferably capable of expanding to a diameter of about 14.8 millimeters, though slightly lesser and greater diameters are also foreseeable. The length over which the wires 32 and nonconducting members 34 extend along the central conductor 22 can vary, with a length of about twenty-five to about thirty millimeters believed to be particularly suitable for the partial prostate ablation procedure of this invention. While two nonconducting members 34 are shown in FIG. 3 as angularly spaced about 80 degrees apart, it is foreseeable that various numbers and spacing of the members 34 could be used.

In describing the nonconducting members 34 and cap 36, the term “nonconducting” is defined herein as meaning a dielectric, such that a current applied to the central conductor 22 will not flow at any significant level when a RF electrosurgical current is applied by a conventional electrosurgical generator. Furthermore, the term “flexible” is meant to convey that the wires 32 and nonconducting members 34 are able to flex in the manner shown in FIGS. 1 through 3, or the functional equivalent. With respect to the nonconducting members 34, “flexible” does not require that the use of a flexible material. Instead, all that is required is that the nonconducting members 34 are capable of being flexed outward from the central conductor 22, requiring the ability to bend at or near the intersection of the conducting members 34 with the cap 36, bend at some point away from the cap 36 (e.g., the intersection of the conducting members 34 with the sheath 16), and bend or flex continuously or at location(s) of the conducting members 34 therebetween.

Prior to performing ablation, the probe 12 is placed in the prostate while in the stowed (closed) position (FIGS. 4 through 6) and under direct vision with the telescope 18. Patient discomfort is reduced by minimizing the diameter of the sheath 16 and the diameter of the collapsed probe 12, as defined by the nonconducting members 34 and conductive wires 32 when collapsed around the central conductor 22 as shown in FIGS. 4 and 5. Depending on the diameter of the telescope (a typical range of about 2.0 to 2.7 mm), the diameter of the sheath 16 can be, for example, on the order of about 0.236 inch (18 Fr), or about 0.223 inch (17 Fr), or about 0.210 inch (16 Fr), which permit placement of the sheath 16 in the urethra. The collapsed diameter of the probe 12 can be on the order of about 0.6 mm, with greater and lesserdiameters being foreseeable, to permit placement of the probe 12 (including the conductor 22, wires 32, nonconducting members 34, and cap 36) in the prostatic urethra 50.

Once placed, the probe 12 is deployed as shown in FIGS. 1 through 3 to perform the partial ablation procedure, during which RF electrosurgical current (which can be generated by a conventional electrosurgical generator) is conducted through the conductor 22 to the wires 32. As previously noted, deployment occurs through operating the actuation lever 24, which causes the central conductor 22 to retract into the sheath 16, thereby causing the wires 32 and nonconducting members 34 to expand. In a preferred embodiment, the actuation lever 24 is operable as a ratchet, so that the probe 12 can be opened to any one of a number of different deployed positions, each characterized by the wires 32 and nonconducting members 34 being flexed to attain a predeterminable diameter. As such, the probe 12 can be opened to a desired diameter depending on the condition of the prostate. A release 38 is provided to allow the ratchet to be released and the probe 12 collapsed to return to the stowed position of FIGS. 4 through 6.

In comparing FIGS. 1 through 3 and 4 through 6, one can see that expansion of the wires 32 and nonconducting members 34 radially outward away from the central conductor 22 in substantially opposite directions and substantially along their entire lengths, allows for the nonconducting members 34 to be pressed into contact with a duct wall of the prostate, causing the conductive wires 32 to be pressed into contact with the opposing duct wall intended to be treated by ablation.

In a particular partial non-apical ablation procedure performed with the probe 12 of this invention, the probe 12 is expanded so that its wires 32 contact the lower region of the prostatic urethra 50 from about 3:00 to abut 9:00 (on the clock face), while the remainder of the prostatic urethra 50, including the upper (apex) region, is held apart from the lower region by the nonconducting members 34. Thereafter, partial transurethral ablation of the prostate (TUAP) is performed by conducting RF current through the central conductor 22 is the wires 32, which ablate the lower region of the prostatic urethra 50, generally between 2:30 and 9:30 (on the clock face), while ablation of the upper (apex) region between 10:00 and 2:00 (on the clock face) is avoided. According to a preferred aspect of the invention, partial transurethral ablation of the prostate in this manner avoids damage to the apex of the prostrate, and particularly to the nerves (e.g., the obturator nerve) at the apex. The ablation device 10 is able to perform controlled partial electrosurgical ablation in a single procedure selectively on the radial area between 2:30 and 9:30, (on the clock face), of the prostatic urethra 50 inferior of the apex, and no ablation between 10:00 and 2:00 (on the clock face), contrary to ablation and resection procedures previously performed with prior art devices. As such, the partial, non-apical prostate ablation procedure of this invention is contrary to the conventional wisdom of using total ablation procedures to treat BPH. Instead of both the lower and upper (apex) regions being ablated, only the lower region is ablated.

Different insertion methods can be employed to place the probe 12 for transurethral ablation of the prostate (TUAP). A first entails inserting an introducing device, e.g., a guide wire, which is first passed through the urethra into the bladder. The probe 12 is then positioned over the guide wire and, under direct vision, the probe 12 “follows” the guide wire into the prostatic urethra 50 and the bladder. Once bladder access has been visually confirmed, the guide wire is removed by pulling it proximally toward the surgeon and out of the device 10.

A second method entails inserting an introducing device 40, for example, a stylet or obturator, through the internal flow channel of the central conductor 22 and out through an opening 44 in the cap 36 at the end of the conductor 22, as represented in FIGS. 4 through 6. The introducing device 40 has a curved distal end 42 with elastic memory such that it protrudes from the cap 36 in a curved condition when pushed forward (distally), and when retracted or pulled backwards (proximally) the curve is elastically deformed such that it is straight and contained within the internal flow channel of the conductor 22. The probe 12 and sheath 16, with the introducing device 40 retracted (straight position), is inserted through the urethra and under direct vision, and the introducing device 40 is then extended (curved) or retracted (straightened) as necessary to gain access to the prostatic urethra 50 and the bladder. Once bladder access has been visually confirmed, the introducing device 40 can be removed by pulling it proximally toward the surgeon and out of the device 10 through the irrigation fluid port 28. Thereafter, irrigation fluid can be introduced through the port 28 and delivered to the probe 12 via the outlet ports 26 of the conductor 22

From the above, one skilled in the art will realize that the partial prostate ablation device and procedure of this invention are capable of protecting the apex of the prostate, where nerves susceptible to injury are located. As such, the device and procedure greatly reduce the skill level needed for doctors to perform a TURP procedure by protecting against common errors that could lead to injury of the apex and nerves. Another notable feature of the invention is that it can be adapted for use in a doctor's office or in a hospital or surgery center operating room with the advantage of allowing direct vision during placement of the probe 12 and during the ablation process. The device 10 can also be adapted for use in a doctor's office as a standalone self-contained device.

While the invention has been described in terms of a preferred embodiment, it is apparent that other forms could be adopted by one skilled in the art. For example, the physical configuration of the probe 12 and device 10 could differ from that shown, and materials and processes other than those noted could be used. Therefore, the scope of the invention is to be limited only by the following claims.

1. An ablation device configured for performing partial non-apical transurethral ablation of the prostate, the ablation device comprising:
a sheath;
a first conductor member reciprocably extending from a distal end of the sheath, the first conductor member having a distal end that extends distally from the distal end of the sheath;
a nonconducting cap disposed at the distal end of the first conductor member, the nonconducting cap having a distal opening therein;
at least two nonconducting members interconnecting the nonconducting cap and the distal end of the sheath; and
multiple flexible conductor members distally extending from the distal end of the sheath, along the first conductor member, and interconnected with the distal end of the first conductor member;
wherein the first conductor member defines a central channel configured to enable an introducing device to be passed therethrough and out through the distal opening of the nonconducting cap;
wherein the device has a stowed position in which the first conductor member extends from the sheath and the nonconducting members and the flexible conductor members are parallel to the first conductor member;
wherein the sheath is sized to be inserted into the urethra and in the stowed position the first conductor member, the nonconducting cap, the nonconducting members, and the flexible conductor members are sized to be inserted into the prostatic urethra;
wherein the sheath, the first conductor member, the nonconducting members, and the flexible conductor members are interconnected so that retraction of the first conductor member relative to the sheath causes the distal end of the first conductor member to move proximally toward the sheath and causes the nonconducting members and the flexible conductor member to expand radially outward and away from the first conductor member and away from each other; and
wherein, when the first conductor member is retracted to expand the nonconducting members and the flexible conductor member, the flexible conductor member is operative to perform ablation of a lower region of the prostatic urethra between 2:30 and 9:30 (on the clock face) while an upper (apex) region of the prostatic urethra between 10:00 and 2:00 (on the clock face) is held apart from the lower region by the nonconducting members so that the upper (apex) region does not undergo ablation.

2. The ablation device according to claim 1, wherein the device comprises seven of the flexible conductor members.

3. The ablation device according to claim 1, wherein the first conductor member has an outlet port fluidically connected to the internal channel thereof, and the internal channel and the outlet port are operative to cause a fluid flowing through the internal channel to be discharged from the first conductor member through the outlet port.

4. The ablation device according to claim 1, wherein the distal opening of the nonconducting cap and the internal channel of the first conductor member are sized and located to enable a guide wire, stylet, and/or obturator to pass therethrough to extend distally beyond the nonconducting cap.

5. The ablation device according to claim 1, wherein the device is in a deployed position and the nonconducting members and the flexible conductor members are elastically displaced radially outward along substantially their entire lengths when the first conductor member is retracted relative to the sheath.

6. The ablation device according to claim 1, wherein the device further comprises ratchet means for retracting the first conductor member relative to the sheath, wherein the device has a plurality of deployed positions.

7. The ablation device according to claim 1, further comprising a channel disposed within the sheath and sized to receive a telescope therein.

8. The ablation device according to claim 1, further comprising an introducing device within the central channel and extending distally through the distal opening in the nonconducting cap.

9. The ablation device according to claim 1, wherein the introducing device is a guide wire, stylet, and/or obturator.

10. The ablation device according to claim 1, wherein when expanded the device has an outer diameter defined by the first conductor member, the nonconducting members, and the flexible conductor members that is about 14.8 millimeters or less.

11. A procedure for performing partial non-apical transurethral ablation of the prostate, the procedure comprising the steps of:
inserting an ablation probe within the prostatic urethra of the human body with the assistance of an introducing device; and then causing a current to flow through multiple conductor members of the ablation probe to perform a controlled and selective electrosurgical ablation of a lower region of the prostatic urethra between 2:30 and 9:30 (on the clock face) and not an upper region of the prostatic urethra between 10:00 and 2:00 (on the clock face) by holding the upper region apart from the lower region with at least two nonconducting members through which current does not flow.

12. The procedure according to claim 11, wherein the upper region is the apex of the prostate.

13. The procedure according to claim 11, wherein the ablation probe is a partial prostate ablation probe comprising a sheath, a first conductor member, the nonconducting members, the flexible conductor members, and a nonconducting cap disposed at a distal end of the first conductor member.

14. The procedure according to claim 13, wherein the first conductor member is supported with and reciprocable relative to the sheath, the first conductor member has a distal end that distally extends beyond the sheath, the flexible conductor members are supported with the sheath, extend along the first conductor member, and are interconnected with the first conductor member, and the nonconducting cap is interconnected with the nonconducting members to the sheath.

15. The procedure according to claim 14, and wherein the first conductor member, the nonconducting members, the flexible conductor members, and the nonconducting cap are all placed within the prostatic urethra during the procedure.

16. The procedure according to claim 15, further comprising the step of expanding the nonconducting members and the flexible conductor members outward from the first conductor member in substantially opposite directions so that the nonconducting members contacts the upper (apex) region of the prostatic urethra, the flexible conductor members contact the lower region of the prostatic urethra from about 3:00 to about 9:00 (on the clock face), and the current flows through the flexible conductor members to ablate the tissue of the lower region of the prostatic urethra between 2:30 and 9:30 (on the clock face).

17. The procedure according to claim 16, wherein when expanded the probe has an outer diameter defined by the first conductor member, the nonconducting members, and the flexible conductor members that is about 14.8 millimeters or less.

18. The procedure according to claim 11, wherein the introducing device is a guide wire and the ablation probe is inserted into the prostatic urethra by placing the guide wire in the urethra and using a central channel within the ablation probe to cause the ablation probe to follow the guide wire.

19. The procedure according to claim 11, wherein the introducing device is an obturator or stylet and is placed in a central channel within the ablation probe before the ablation probe is inserted into the prostatic urethra.
